(19) United States
(12) Patent Application Publication
(10) Pub. No.: US 2024/0268671 A1
(43) Pub. Date: Aug. 15, 2024

(54) MEDICAL PALPATION DEVICE, SYSTEM AND METHOD FOR AUGMENTED PHYSICAL EXAMINATION

(71) Applicant: MedAmerica Consulting Employees, Inc. dba Inflect Health, Emeryville, CA (US)

(72) Inventors: Joshua Tamayo-Sarver, Los Gatos, CA (US); Samuel Tamayo-Sarver, Los Gatos, CA (US); Isaac Tamayo-Sarver, Los Gatos, CA (US)

(73) Assignee: MedAmerica Consulting Employees, Inc. dba Inflect Health, Emeryville, CA (US)

(21) Appl. No.: 18/442,057

(22) Filed: Feb. 14, 2024

Related U.S. Application Data
(60) Provisional application No. 63/445,997, filed on Feb. 15, 2023.

(51) Int. Cl. A61B 5/00; A61B 90/00; G16H 50/20; G16H 50/30

(52) U.S. Cl. CPC A61B 5/0053; A61B 5/002; A61B 5/7271; A61B 5/742; A61B 90/06; G16H 50/20; G16H 50/30; A61B 2090/064; A61B 2560/0406; A61B 2560/045; A61B 2560/0462; A61B 2562/0247

(57) ABSTRACT

A handheld palpation device for remote medical examination comprises a handle to apply pressure to a patient's body using a palpation head. The handle includes an interior space and an opening to the interior space. The palpation head fits inside the opening on one end, and the other end comprises a round pliable head configured to contact the body of the patient when pressure is applied. A pressure pad positioned in the interior space derives electrical pressure data when pressure is applied. A sensor chip in the interior space receives the electrical pressure data from the pressure pad, calculates one or more quantitative pressure measurements, and transmits the quantitative measurements to a communications chip connected to the sensor chip which communicates the quantitative pressure measurements to an external device for analysis using machine learning to provide a clinical assessment using the pressure measurements.

### CROSS REFERENCE TO RELATED APPLICATIONS

This application claims the benefit of U.S. Provisional Application No. 63/445,997, filed on Feb. 15, 2023. The entire content of these application is hereby incorporated herein by reference.

### FIELD OF THE INVENTION

This disclosure relates generally to virtual or remote care in medicine, and more specifically remote or augmented physical examination of a patient using a medical palpation device held in the patient's hand and used by the patient at a clinician's direction to palpate a patient's body during a virtual physical examination.

### BACKGROUND

Abdominal pain is the most common reason for unscheduled medical care. According to the American Hospital Association, there are over 130 million emergency department (ED) visits per year. See American Hospital Association, Fact Sheet: Telehealth, February 2019. Eleven million ED visits have a principal reason of stomach and abdominal pain, cramps, and spasms. Of those, six million receive a primary diagnosis in the abdominal pain group. See Reed M E, Parikh R, Huang J, et al., Real-Time Patient-Provider Video Telemedicine Integrated with Clinical Care, N Engl J Med. 2018; 379:1478-1479.

The Centers for Medicare & Medicaid Services has produced guidelines for documenting in-person physical exam guidelines by twelve organ systems or seven body areas. See Centers for Medicare & Medicaid Services, 1995 Documentation Guidelines for Evaluation and Management Services, and 1997 Documentation Guidelines for Evaluation and Management Services.

There are an extensive number of diagnoses associated with abdominal pain. Palpating the abdomen enables a clinician to determine if there is a chance of “surgical abdomen,” which is pathology that may need surgical intervention, such as appendicitis, small bowel obstruction, intestinal perforation, hernia, etc. When a clinician palpates the abdomen of a patient and based on their palpation, suspects that the patient may have a surgical abdomen, the patient is then further evaluated with advanced imaging: computed tomography scan (CT), ultrasound (US), or magnetic resonance imaging (MRI). Occasionally, the patient will be admitted as an inpatient for repeat abdominal examinations or taken directly to the operating room for surgical management without advanced imaging.

Ten million total CT scans are performed on the abdomen or pelvis annually. See Reed et al. Furthermore, although CT use is common in patients that present to the ED with abdominal pain (˜40% for adults, ˜15% for pediatric), CT use is linked with some significant risk, including higher rates of cancer. See Centers for Medicare & Medicaid Services, 1995 and 1997 Documentation Guidelines for Evaluation and Management Services.

Telehealth has been an increasingly important aspect of the delivery of healthcare. According to the American Hospital Association, telehealth has the potential to reduce costs and improve access to care, especially for underserved populations and residents of rural areas. The American Hospital Association fact sheet reports that 79% of hospitals in the United States use some form of telehealth. Despite the growth in telehealth, abdominal pain generally cannot be safely and accurately evaluated virtually because the clinician cannot palpate the abdomen to determine if a patient needs advanced imaging or surgery.

For telehealth to be maximally beneficial, patients need to have as much of the tasks performed at an in-person physician visit done virtually. A complete Virtual Physical Exam (VPE) allows a clinician to conduct an exam remotely. In order for a clinician to perform a physical examination effectively in a telehealth visit, a VPE should provide real-time, high-quality information similar to an in-person visit. In addition to reliance on videoconferencing applications for telehealth visits, episodic use from inexpensive devices may be needed. In particular, a virtual physical examination should include objective data from and needs to enable the following tasks:
Observe/inspect (General, Eyes, Ears, Throat, Skin, Injury)
Auscultate (Heart, Lungs, Neck, Abdomen)
Palpate (Injured site, Lymph nodes, Abdomen, Calves, skin)
Get Vital Signs (Heart rate, Respiratory rate, Blood pressure, Pulse oxygen saturation, Weight, Height)
   Although there are many technological options available for remote inspection (visual), auscultation (audio), and getting vital signs for telehealth visits, there remains a paucity of methods and information available with regards to palpation during telehealth visits. For example, U.S. Pat. No. 9,826,904 “System and method rejecting tissue surface properties” provided a system to identify the shape of the body or part, but does not assess for clinical tenderness. In another example, U.S. Pat. No. 6,192,143 “Apparatus for detecting very small breast anomalies” mechanically palpates a breast surface to determine small lumps or other anomalies, but does not provide information on tenderness. In addition, U.S. Pat. No. 5,351,677 “Medical system having objective information reproduction means for palpation,” provides a means for an operator to remotely palpate distant objects with tactile feedback, but does not enable the patient or caregiver to provide self palpation and useful information to a clinician.

Notably, palpation in abdominal exams continues to lack an alternative to patient-reported and subjective descriptions of the patient's experience with self-examination and palpation. This may contribute to gastroenterology ranking as the second lowest internal medicine specialty to incorporate telemedicine per the American Medical Association's Patient Practice Benchmark Survey. Thus, while telehealth does enable clinicians to care for many clinical conditions remotely, it does not readily allow for the diagnosis and management of ailments such as acute abdominal pain where the clinician needs to ascertain if the patient has a “surgical abdomen” and needs advanced imaging, lymph node concerns, traumatic injuries, or musculoskeletal complaints, all of which generally require the ability to palpate the abdomen or other area of injury/pain.

### SUMMARY

A device, system, and method to perform standardized remote home palpation will enable safe virtual care of abdominal pain patients and provide an avenue for health care providers to increase the scope of pathologies that may be safely and accurately assessed via telemedicine.

Embodiments of a physical examination device, system and method disclosed herein enable tele-palpation of a patient by standardizing home palpation in order to enable safe virtual care of abdominal pain patients. The device enables a patient or caregiver to palpate (press) on the patient's abdomen or area of concern and provide sensor pressure data to a software product connected to the device during palpation of the abdomen. Embodiments of a software product disclosed herein analyze the sensor pressure data and provide usable data to a clinician regarding risk of the patient's abdominal pain, including information regarding whether advanced imaging is likely needed. The clinician may then incorporate the device's findings into the clinician's decision-making about the likelihood of pathology (appendicitis, a broken bone, etc.).

In one embodiment, a handheld device is intended for use by patients during virtual abdominal exams and other virtual physical exams where palpation is needed. Embodiments of the disclosed palpation device include a non-sterile, reusable medical device that can provide data and information to a remote healthcare provider to help determine whether abdominal imaging or further in-patient treatment in the ED is needed.

In some embodiments, the device is disclosed as comprising a handle, a palpation head, a digital force sensor and processing circuit, a compression member comprising a comfort spring or rigid block, a rechargeable battery pack, a Bluetooth module, and a USB port. During use, the patient or caregiver takes the device and pushes on their sternum as hard as they can or until they experience any discomfort to establish a baseline. The device measures the maximum force applied and transmits it via Bluetooth to a connected computer or smartphone. The connected computer or smartphone has a software application that receives the force data, applies experimentally derived algorithms to the data, and then informs the clinician of the extent to which the patient has clinically significant tenderness. The clinician can then integrate this information with the history and other physical exam findings to arrive at a diagnosis.

### BRIEF DESCRIPTION OF THE DRAWINGS

FIG. 1 illustrates a front external view of a medical palpation device for remote physical examination in accordance with an embodiment of the present disclosure.

FIG. 2 illustrates a cross-sectional view of a medical palpation device for remote physical examination showing a cord connection in accordance with an embodiment of the present disclosure.

FIG. 3 illustrates a top left perspective view of a medical palpation device for remote physical examination in accordance with an embodiment of the present disclosure.

FIG. 4 illustrates a top left perspective exploded view of a medical palpation device for remote physical examination in accordance with an embodiment of the present disclosure.

FIG. 5 illustrates a cross sectional view of a medical palpation device for remote physical examination in accordance with an embodiment of the present disclosure.

FIG. 6 illustrates a top left perspective exploded view of a medical palpation device for remote physical examination in accordance with an embodiment of the present disclosure.

FIGS. 7A-7F illustrate exemplary screenshots of a patient software application for use with a medical palpation device for remote physical examination in accordance with an embodiment of the present disclosure.

FIG. 8 illustrates an exemplary flowchart showing operations for a data analytics software process in accordance with an embodiment of the present disclosure.

FIGS. 9A-9C illustrate exemplary wireframe diagrams depicting screenshots showing an exemplary user interface for a clinician treating a patient using a medical palpation device for remote physical examination in accordance with an embodiment of the present disclosure.

FIG. 10 shows an example of a computer system, one or more of which may be used to implement one or more of the apparatuses, systems, and methods illustrated herein.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

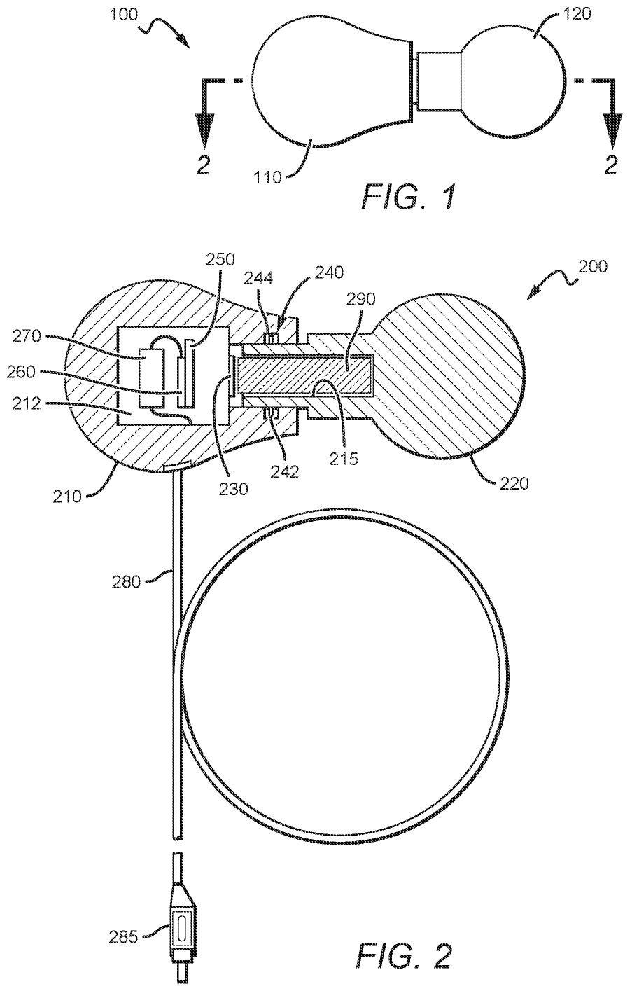

FIG. 1 illustrates a front external view of a medical palpation device 100 for remote physical examination in accordance with an embodiment of the present disclosure. The palpation device shown in FIG. 1 comprises an ergonomic handle 110 having one end that is round and shaped to comfortably be held in a patient's or caregiver's hand during the pushing or pressing movements of abdominal palpation. In some embodiments, the ergonomic design is optimized for both comfort and structure. A comfortable handle means that a patient will be more likely to use it consistently to produce accurate results. A structural aspect of the handle permits the device to support sufficient surface area for a moderate amount of force to be applied by the user without compromising the structural integrity of the handle. The structure of the handle also keeps the space between the handle, sensor, and massage head substantially constant with sufficient room inside the handle for internal circuitry. In some embodiments, the handle is ergonomically designed rigid construction, and may be comprised of materials such as a rigid smooth plastic or polyvinyl carbonate material. Other materials such as wood or leather may even be used. The interior of the handle may be hollow or otherwise have an internal space to allow for containment or housing of chips, pressure pads, and other components as described below.

A second end of the palpation device comprises an opening (115, 215 as shown in FIGS. 2, and 415 as shown in FIG. 4, for example) to the interior space of the handle. The device 100 further comprises a palpation head 130, wherein one end of the palpation head 130 is configured to fit inside the opening of the handle 100, and the other end comprises a round pliable head configured to contact the patient's body when pressure is applied by a user's hand. In some embodiments, the palpation head 130 comprises a massage head similar to those used in handheld electronic massage devices or electronic massager kits. In one embodiment, palpation head 130 comprises a massage head having a firm foam ball that provides an optimal balance of friction with the body part being palpated, body part contour conforming properties, and firmness for pressure transmission. The palpation head 130 may comprise a massage head that provides a comfortable foam-like surface that provides compression over a surface area to provide better palpation and consistency when a user holds and applies the palpation device to a patient's body.

FIG. 2 illustrates a cross-sectional view 200 of an embodiment of medical palpation device 100 for remote physical examination showing a cord connection, showing handle 210 with internal space 212 and opening 215. Pressure pad 230 resides inside internal space 212 and uses electrical resistance to capture the pressure applied by the user. In some embodiments, the pressure pad 230 comprises a force sensor. In one embodiment, the force sensor comprises a force sensitive resistor. In some embodiments, the force sensor is calibrated to a range of force known to be applied by people when self-palpating to provide the greatest force discrimination in a relevant force range.

Lock system 240 in FIG. 2 resides within opening 215 and functions to lock the palpation head 220 (e.g., massage head) in place within handle 210 to prevent ejection of the palpation head from the palpation device during use. Lock system 240 comprises, in some embodiments, a horizontal bar or pin 242 that fits into vertical slots 244 inside opening 215. In one embodiment, the vertical slots allow the horizontal bar 242 approximately 4 mm of travel between the massage head and the handle to accomplish an optimal handling or feel for the operation of the palpation device during the palpation process.

Sensor internal chip 250 as shown in FIG. 2 is connected in one embodiment to pressure pad 230. Pressure pad 230 is connected by wires to sensor internal chip 250. In one embodiment, sensor internal chip 250 comprises an internal circuit board chip that translates the electrical signals from the pressure pad into quantitative measurements that can be transmitted to a networked device via a voltage divider circuit 260 which may relay data from the sensor internal chip 250, through cord connection 280 and eventually over the internet to a clinician for interpretation as appropriate. In some embodiments, software code may be used to measure and record the electrical resistance and turn it into pounds of force. In one embodiment, such software code is manufactured by Tekscan, Inc. under the Flexiforce™ trade name.

In some embodiments, the voltage divider circuit 260 comprises an analog circuit module that processes the external force applied by the user. Voltage divider circuit 260 may have a programmable reference voltage for sensitivity adjustment, and a connection to pressure pad 250 (e.g., a force sensitive resistor). In one embodiment, pressure pad 250 comprises a force sensitive resistor A201-25 pad manufactured by Tekscan, Inc. under the trade name Flexiforce™. In one embodiment, a voltage divider circuit 260 may comprise a voltage divider circuit module also manufactured by Tekscan, Inc. Voltage divider circuit 260 may have a programmable reference voltage to set a default sensitivity adjustment to calibrate the force sensor. In other embodiments, an inverting op-amp circuit module or a non-inverting op-amp circuit module (not shown) may be used in place of voltage divider circuit module 260. In some embodiments, internal space 212 of handle 210 in FIG. 2 is of sufficient size such that any interference of voltage divider circuit 260 that may be caused by interference of skin, metal or any other ionized surface with the open circuit of voltage divider 260 will not cause a problem by impacting the force readings.

In some embodiments, cord connection 280 comprises a cord sheath that protects the wires from damage inside the handle, and a power cord to connect the palpation device to a power adapter or charger, and/or a user device such as mobile phone, tablet or laptop. In some embodiments, cord connection 280 is connected to power source 270 which may comprise a rechargeable battery known in the art such as a lithium-ion battery. In one embodiment, power source 270 may also include a power transfer module comprising a small chip that enables transfer of power and data from the palpation device to a user device such as a mobile phone, tablet or laptop or desktop computer. Cord connection 280 may also comprise a USB-C port in one embodiment, which allows for recharging of the palpation device, powering the device from an external charger, and direct connection to a computer for data transfer. Cord connection 280 may also comprise a power cord that is connected to one or more connector heads 285 that may comprise one or more connectors such as a micro-USB, Lightning port connector, or USB-C port connector that may connect to a user's mobile phone, tablet, laptop computer, or other networked device.

Finally, in the embodiment shown in FIG. 2, sensor deformer 290 is positioned inside opening 215 and also connected to palpation head 220 to provide the contact point where the palpation head pushes against the pressure pad 230.

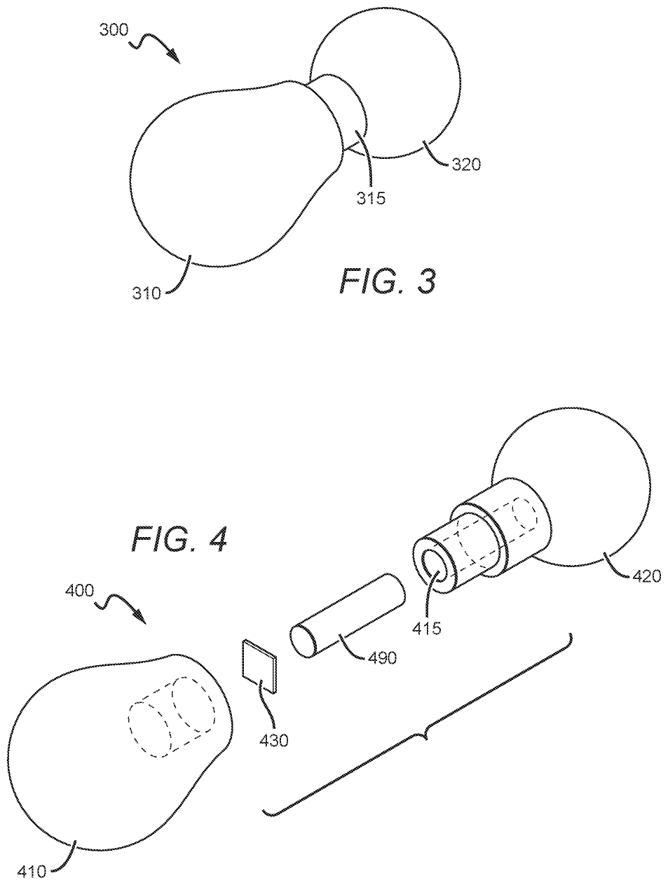

FIG. 3 illustrates a top left perspective view 300 of a medical palpation device for remote physical examination in accordance with an embodiment of the present disclosure. This external view shows handle 310, palpation head 320, and an area where opening 315 connects the handle 310 with one end of palpation head 320.

FIG. 4 illustrates a top left perspective exploded view 400 of a medical palpation device for remote physical examination in accordance with an embodiment of the present disclosure. The exploded view illustrates the relative positions of certain components of the embodiments of the medical palpation device show in FIGS. 1,2, and 3, including ergonomic handle 410, pressure pad 430, sensor deformer 490, and palpation head 420.

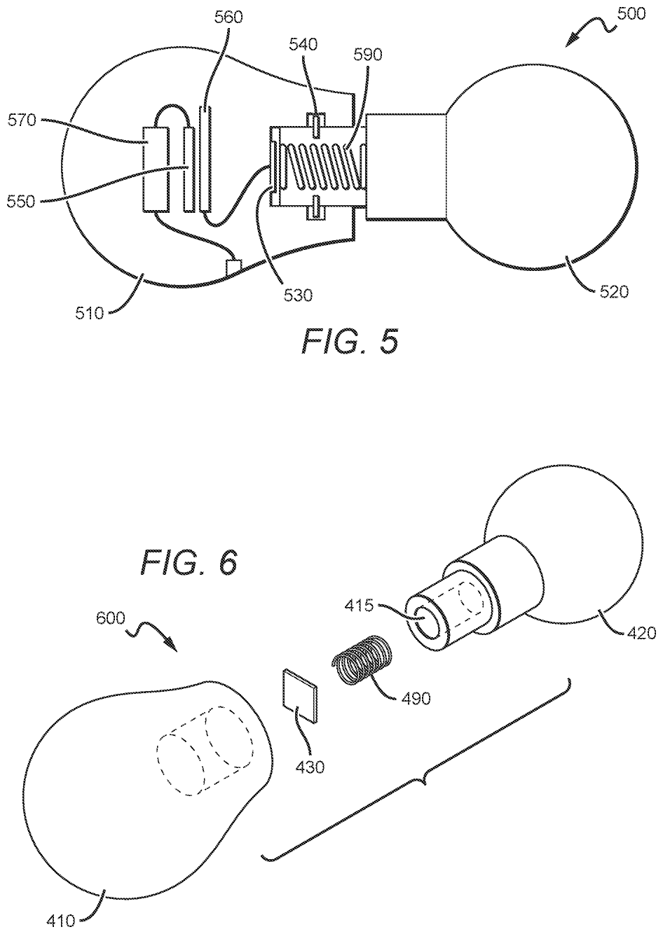

FIG. 5 illustrates a cross sectional view of a medical palpation device 500 for remote physical examination in accordance with an embodiment of the present disclosure. FIG. 5 shows components analogous to those described above in FIGS. 1-4. Such analogous components include an ergonomic handle 510 as a simple round, ergonomically shaped handle having interior space for chips and other components, palpation head (e.g., massage head) 520 for pressing against the patient's body, pressure pad 530 for generating data from the amount of pressure applied, lock system 540 for locking the palpation head in place to prevent ejection of the palpation head during use, and sensor internal chip 550 which is connected in one embodiment to pressure pad 530. Pressure pad 530 is connected by wires to sensor internal chip 550. In one embodiment, sensor internal chip 550 comprises an internal circuit board chip that translates the electrical signals from the pressure pad 530 into quantitative measurement data that can be transmitted to a networked device via a Bluetooth internal chip 560, which connects and transmits the data from the pressure pad 530 to a device such as a Bluetooth connected computer, tablet or smart phone.

In some embodiments, power port 580 is connected to an internal power source 570 for powering pressure pad 530, sensor internal chip 550, and Bluetooth internal chip 560. Power source 570 may in one embodiment comprise a rechargeable battery known in the art such as a lithium-ion battery. Power port 580 may further comprise a data port, and/or a power cord connection that includes a USB-C port in one embodiment, which allows for recharging of the palpation device, powering the device from an external charger, and/or direct connection to a computer for data transfer. Power port 280 may also comprise a power cord that is connected to one or more connector heads 285 that may comprise one or more connectors such as a micro-USB, Lightning port connector, or USB-C port connector that may connect to a power source such as an electrical outlet or power bank, or a user's mobile phone, tablet, laptop computer, or other networked device.

In the embodiment shown in FIG. 5, a spring 590 is positioned in between the ergonomic handle 510 and the palpation head 520 to apply pressure to the palpation head 520 to extend it out into the patient's body part (e.g., the patient's abdomen) during palpation. In some embodiments, use of spring 590 permits a softer and more natural feel of the palpation.

FIG. 6 illustrates a top left perspective exploded view 600 of the medical palpation device for remote physical examination shown in FIG. 5 in accordance with an embodiment of the present disclosure. The exploded view illustrates the relative positions of certain components of the embodiments of the medical palpation device show in FIG. 5, including ergonomic handle 610, pressure pad 630, spring 690, and palpation head 620.

Embodiments of the medical palpation devices shown in FIGS. 1-6 may be configured to use with interpretation software as described below. The interpretation software comprises desktop or mobile applications that include software that resides on the computer or smartphone that connects with the palpation device, guides the user through the palpation movements, receives the sensor data, and then applies data processing algorithms to determine the extent to which the sensor data indicates that the patient has clinically significant tenderness.

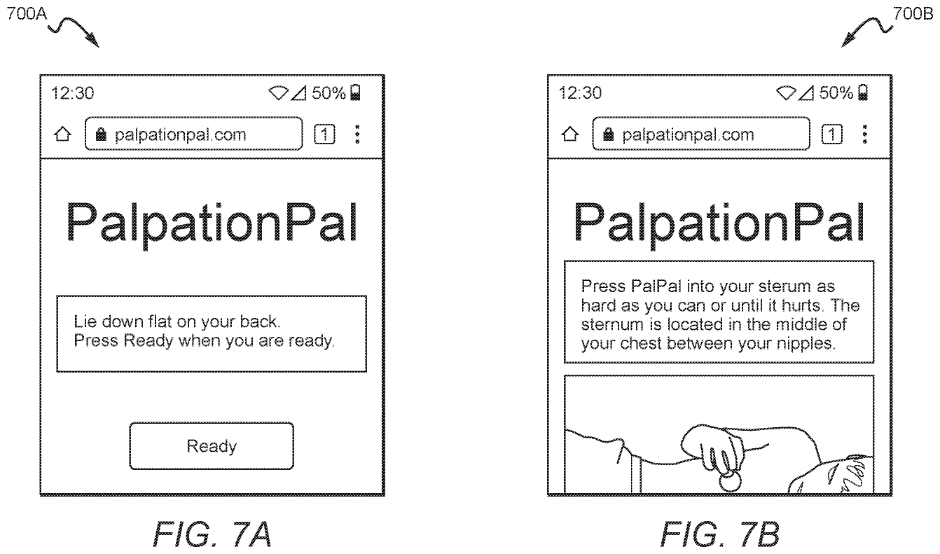

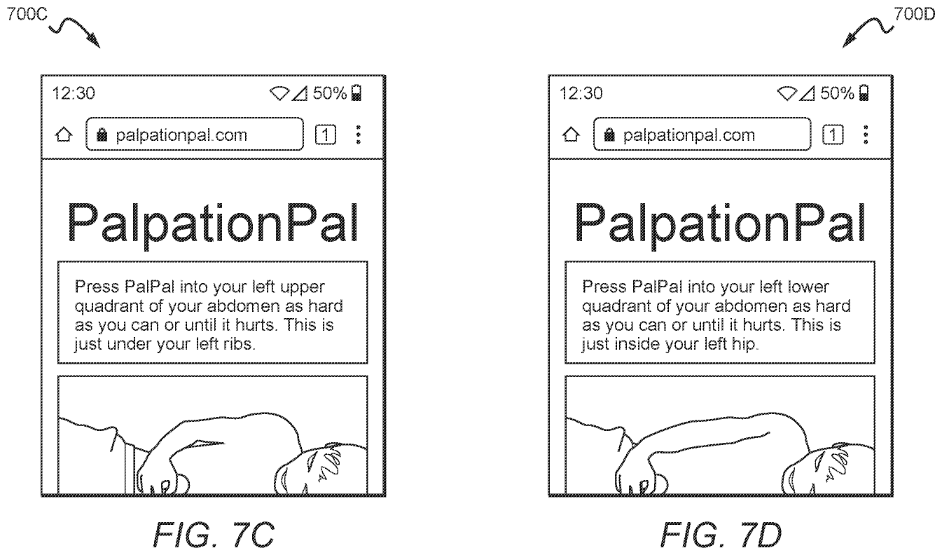

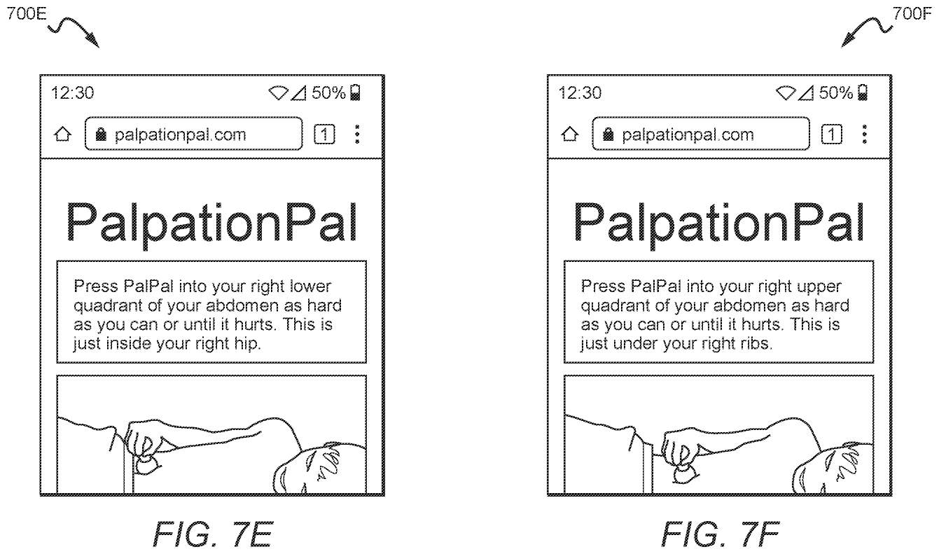

FIGS. 7A-7F illustrate exemplary screenshots of a patient software application for use with a medical palpation device for remote physical examination in accordance with an embodiment of the present disclosure. The screenshots in FIGS. 7A-7F show a user demonstration sequence for the medical palpation device of some embodiments of the present disclosure. Prior to starting the user demonstration sequence in some embodiments, a patient may login on their networked computing device, e.g., mobile phone, tablet, or laptop, for a virtual visit with their telehealth clinician or care provider. In one example, the patient may have identified abdominal pain as a symptom. The telehealth care provider may the issue a preliminary instruction to the patient to go and get the medical palpation device and turn it on. If needed, the palpation device should be plugged into a power source. The palpation device should also be connected to the networked device via a physical cord or a Bluetooth connection to transmit the pressure sensor data readings to the interpretation software program for assessing the clinical significance of the abdominal or other pain of the patient.

Once these preliminary steps have been completed, in some embodiments of the present disclosure a web or mobile application launches on the networked device (e.g., smart phone, tablet, or laptop/desktop computer). After the patient clicks “Accept”, a demonstration sequence guides the patient or caregiver to follow the directions in the user demonstration sequence for palpating the patient's body. A patient may perform the sequence on his or her own body using the palpation device for self-palpation. Alternatively, a caregiver may perform the palpation sequence by applying the palpation device to a patient's body. In one embodiment, the demonstration sequence guides the patient or caregiver to palpate a patient's abdomen. In screenshot 700A shown in FIG. 7A, the patient is first instructed to lie down flat and click the “Ready” button when ready to proceed with the palpation. Screenshots 700A-700F in FIGS. 7A-7F also show how a user may hold or grip the palpation device in order to perform the palpation sequence as instructed.

First, in screenshot 700B shown in FIG. 7B, the user is instructed to palpate the patient's sternum. The user is instructed to press the palpation device into the patient's sternum as hard as the patient (or user) can until it hurts. The patient is instructed that the sternum is located in the middle of the chest between the patient's nipples.

Next, the patient or user is requested to identify the four abdomen quadrants to be palpated: Left Upper Quadrant (LUQ), Left Lower Quadrant (LLQ), Right Lower Quadrant (RLQ), and Right Upper Quadrant (RUQ). In screenshot 700C shown in FIG. 7C, the user is instructed to palpate the Left Upper Quadrant (LUQ) by pressing the palpation device into the left upper quadrant of the patient's abdomen as hard as possible or until it hurts. The LUQ is just under the patient's left ribs. In screenshot 700D shown in FIG. 7D, the user is instructed to palpate the Left Lower Quadrant (LLQ) by pressing the palpation device into the left lower quadrant of the patient's abdomen as hard as possible or until it hurts. The LLQ is located just inside the patient's left hip. In screenshot 700E shown in FIG. 7E, the user is instructed to palpate the Right Lower Quadrant (RLQ) of the patient's abdomen as hard as possible or until it hurts. The RLQ is located just inside the patient's right hip.

Next, the patient or user is instructed to palpate the Right Upper Quadrant (RUQ) of the patient's abdomen as hard as possible or until it hurts, as shown in screenshot 700F of FIG. 7F. the RUQ is located just under the patient's Right ribs. Once the palpation of screenshot 700F is completed, the user may press “Submit” to transmit the pressure sensor data and run the data analytics software for transmission to the clinician.

It is expected that the patient may have a mechanism to begin measurements and stop measurements when the patient indicates that they are finished pushing on the designated location in each palpation. In some embodiments, the medical palpation device produces five data files for the patient containing pressure sensor data from each of the 5 palpations performed on the patient as instructed by the software. Sensor data may be collected multiple times at regular intervals (e.g. 50 times/second) during the palpation. In one embodiment, for each of the five sensor data files, the average of the top 25 maximal pressure readings are used as discussed below. This produces one pressure reading for each of the five palpation locations on the patient's body. For each of the four abdominal quadrants (LUQ, RUQ, LLQ, RLQ), in one embodiment the difference between the pressure reading and the baseline sternum pressure reading is used as the maximum reliable pressure for that quadrant. This data is then transmitted from the palpation device to the patient's mobile device, tablet, and/or laptop or desktop computer for further analysis.

In one example use case of embodiments of the medical palpation device and data interpretation software discussed herein, a 27-year-old woman is experiencing abdominal pain and would like to have a medical assessment. She contacts her physician's office, who arranges a virtual visit. Before the virtual visit, the woman uses other devices to assess her vital signs (temperature, heart rate, respiratory rate, blood pressure, and pulse oxygen saturation) and provide recordings for physician review of her heart, lungs, and abdomen. She then uses the medical palpation device discussed herein with respect to various embodiments to push on the four quadrants of her abdomen, which provides the data to the physician to use during the encounter. The physician starts the virtual visit with the woman. Based on the woman's history and the device's indication that she has clinically significant tenderness in the right lower quadrant, the physician orders a same-day ultrasound of the woman's appendix to be performed to evaluate for appendicitis.

In another example use case of embodiments of the medical palpation device and data interpretation software discussed herein, a 30-year-old man is experiencing nausea and vomiting and would like to have a medical assessment. He logs into virtual urgent care. Before the virtual visit, the man uses other devices to assess her vital signs (temperature, heart rate, respiratory rate, blood pressure, and pulse oxygen saturation) and provide recordings for physician review of his heart, lungs, and abdomen. He then uses this invention to push on the four quadrants of his abdomen, which provides the data to the physician to use during the encounter. The physician starts the virtual visit with the man. Based on the man's history and the device's indication that there is no clinically significant tenderness in the abdomen, the physician recommends an antiemetic, water and Gatorade for likely gastroenteritis.

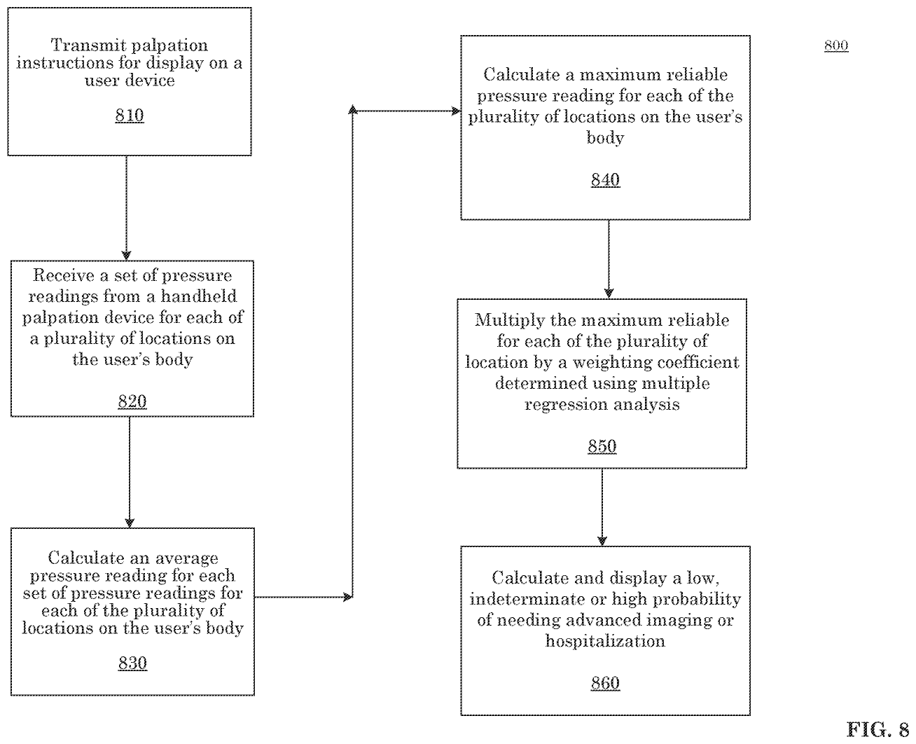

FIG. 8 illustrates an exemplary flowchart 800 showing operations for a data analytics software process in accordance with an embodiment of the present disclosure. First, the system in step 810 transmits palpation instructions for display to a user on a user device such as a smart phone, tablet, or desktop or laptop computer. An exemplary interface for this is presented and discussed above with respect to FIGS. 7A-7F above.

In operation 820, a set of pressure readings is received from the handheld palpation device for each of a plurality of locations on the user's body to identify a maximum reliable pressure for each abdominal quadrant as follows. In some embodiments of the invention, operation 820 may be performed in accordance with Step 1 below. Operation 830 may be performed in accordance with Step 2 below. Operation 840 may be performed in accordance with Step 3 listed below. Operation 850 may be performed in accordance with Step 4 listed below. Next, operation 860 may be performed in accordance with Step 5 listed below.

Step 1: Ingest sensor readings for each of the five locations:
i. Sternum
ii. Left upper quadrant
iii. Right upper quadrant
iv. Left lower quadrant
v. Right lower quadrant

Step 2: For each location sensor reading, create maximum pressure by:
i. Trimming the number of pressure sensor data readings to include only the 25 highest readings
ii. Take the average of those 25 highest readings

Step 3: For each maximum pressure in the abdomen, create a maximum reliable pressure as follows:
i. Normalize the abdomen maximum pressure by using the sternum maximum pressure. This is in the form of maximum pressure left lower quadrant (minus) maximum pressure sternum.

Step 4: Multiply the maximum reliable pressure for each of the features and the appropriate weighting coefficient:

i. Coefficient
X LUQ
ii. Coefficient
X LLQ
iii. Coefficient
X RUQ
iv. Coefficient
X RLQ
v. Coefficient
X LUQ X LLQ
vi. Coefficient
X LUQ X RUQ
vii. Coefficient
X LUQ X RLQ
viii. Coefficient
X LLQ X RUQ
ix. Coefficient
X LLQ X RLQ
x. Coefficient
X RUQ X RLQ
xi. Coefficient
X LUQ X LLQ X RUQ
xii. Coefficient
X LUQ X LLQ X RLQ
xiii. Coefficient
X LUQ X RUQ X RLQ
xiv. Coefficient
X LLQ X RUQ X RLQ
xv. Coefficient
X LUQ X LLQ X RUQ X RLQ

Step 5: Combine the above into the predicted score and calculate a probability of needing advanced imaging or hospitalization.

In some embodiments of the invention, a probability of needing advanced imaging or hospitalization is computed using machine learning techniques. In one embodiment an algorithm is developed statistically from approximately 76 patients presenting to a regional medical center (Arrowhead Regional Medical Center (ARMC)) emergency department with a chief complaint of abdominal pain, nausea, vomiting and/or diarrhea. Additional and/or other data may be collected for a training or feasibility set for use in embodiments of the present invention. In some embodiments, a linear regression model is used. Other machine learning models known in the art may also be contemplated in other embodiments. In one embodiment, multiple logistic regression is performed with the outcome variable of the patient being whether the patient requires advanced imaging or admission, and the predictor variables being the maximum reliable pressure on each quadrant with all interaction effects. This produces a predicted probability for admission or advanced imaging, and this is placed into three categories of likelihood: low, indeterminate, and high.

An example implementation in some embodiments is provided below for a representative training data set including a patient sample from a county hospital emergency department as described above.

### Analytics

In one embodiment, each patient in a derivation study had basic data of each patient that consented to participate in the study entered into an Excel spreadsheet: age, birth gender, race ethnicity, height, weight, ED diagnosis, duration of symptoms, disposition, abdominal pain location, final diagnosis, whether or not they required immediate surgical intervention, whether a CT scan was done, and whether an ultrasound was done. Additionally, the palpation device produced five files for each patient containing sensor data. For each of the five sensor data files, the average of the top 25 maximal pressure readings were used. This produced one pressure reading for each of the five locations for the patient, and for the for abdominal quadrant, the difference between that pressure reading and the baseline sternum pressure reading was used as the maximum reliable pressure for that quadrant. Multiple logistic regression was then performed with the outcome variable of the patient required advanced imaging or admission, and the predictor variables where the maximum reliable pressure for each quadrant with all interaction effects. This produced a predicted probability for admission or advanced imaging, and this was placed into three categories of likelihood: low, medium, and high. In one embodiment, the linear regression analyses were performed using Stata 16.1 MP (StataCorp, College Station, TX).

### Results

In one embodiment of a training set, out of 83 patients who consented to participate in the study, there was complete sensor, imaging, and disposition data on 76 patients for our study sample. In this sample of patients that made it back to a bed in a busy County emergency department, there was a very high rate of imaging (86%), admission (42%), and surgical intervention (15%) (table 1). The multivariate logistic regression including the maximum reliable pressure data for each quadrant on the patient that was normalized by the maximum reliable pressure data for the sternum, demonstrated strong modeling characteristics in this derivation study. All interaction terms were included in the model based on an a priori hypothesis and were therefore included in the final model to reduce over fitting the data, although some of the individual interaction terms were not statistically significant (table 2). The model demonstrated a likelihood ratio chi-squared with 15° of freedom of 28.83, a P value of 0.017, and a pseudo-R squared of 0.49. This produced an area under the curve of 0.95.

The purpose of the device is for a provider who is not physically present with the patient to determine whether or not the patient can be safely treated virtually for their abdominal pain. To make this most clinically useful, three categories of risk were created for the patient needing advanced imaging or hospital admission: low, medium, and high. Based on informal clinician feedback, a low risk group was created where the predicted probability was less than 10%, a high risk from group was created where the predicted probability was greater than 90%, and a medium or indeterminate risk group that fell between the two cutoffs. This allows evaluation of the device performance and its intended clinical use, albeit with a derivation set in this case. The performance of the device in this derivation sample was very good without any miscategorizations in either the low or the high risk groups (Table 3).

TABLE 1
Demographics and outcomes of study sample

| | | | | Imaging | |
|---|---|---|---|---|---|
| | Total | CT or | | or | Surgical |
| | N (column | ultrasound | Admission | admission | intervention |
| | %) | N (row %) | N (row %) | N (row %) | N (row %) |
| Overall | 76 (100%) | 65 (86%) | 32 (42%) | 66 (87%) | 11 (15%) |
| Birth Gender | | | | | |
| Female | 45 (59%) | 41 (91%) | 16 (36%) | 41 (91%) | 9 (20%) |
| Male | 31 (41%) | 24 (77%) | 16 (52%) | 25 (81%) | 2 (6%) |
| Age | 42 (55%) | 35 (83%) | 19 (45%) | 35 (83%) | 8 (19%) |
| 18-44 | | | | | |
| 45-65 | 27 (36%) | 25 (93%) | 9 (33%) | 25 (93%) | 2 (7%) |
| Over 65 | 7 (9%) | 5 (71%) | 4 (57%) | 6 (86%) | 1 (14%) |
| Race/ethnicity | | | | | |
| Asian/Pacific | 2 (3%) | 2 (100%) | 1 (50%) | 2 (100%) | 0 (0%) |
| Islander | | | | | |
| Black | 8 (11%) | 5 (63%) | 2 (25%) | 5 (63%) | 0 (0%) |
| Hispanic | 56 (74%) | 49 (88%) | 23 (41%) | 49 (88%) | 9 (16%) |
| Non- | 9 (12%) | 8 (89%) | 5 (56%) | 9 (100%) | 1 (11%) |
| Hispanic | | | | | |
| White | | | | | |
| Other | 1 (1%) | 1 (100%) | 1 (100%) | 1 (100%) | 1 (100%) |
| BMI | | | | | |
| <18.5 | 2 (3%) | 2 (100%) | 1 (50%) | 2 (100%) | 0 (0%) |
| 18.5 to 24.9 | 10 (13%) | 10 (100%) | 5 (50%) | 10 (100%) | 3 (30%) |
| 25 to 29.9 | 13 (17%) | 12 (92%) | 9 (69%) | 12 (92%) | 1 (8%) |
| 30 to 39.9 | 19 (25%) | 18 (95%) | 9 (47%) | 19 (100%) | 3 (16%) |
| >=40 | 7 (9%) | 7 (100%) | 2 (29%) | 7 (100%) | 1 (14%) |
| Missing | 25 (33%) | 16 (64%) | 6 (24%) | 16 (64%) | 3 (12%) |

TABLE 2
multiple logistic regression model with dependent variable-patient
was admitted and/or received advanced imaging

| | | Lower bound | Upper bound |
|---|---|---|---|
| | | 95% confidence | 95% confidence |
| | Odds ratio | interval | interval |
| Right lower quadrant | 0.87 | 0.74 | 1.02 |
| Right upper quadrant | 1.13 | 0.98 | 1.29 |
| RLQ X RUQ | 1.02 | 1.00 | 1.04 |
| LLQ | 1.36 | 1.03 | 1.79 |
| RLQ X LLQ | 1.00 | 1.00 | 1.00 |
| RUQ X LLQ | 0.98 | 0.96 | 1.00 |
| RLQ X RUQ X LLQ | 1.00 | 1.00 | 1.00 |
| LUQ | 0.93 | 0.81 | 1.07 |
| RLQ X LUQ | 0.99 | 0.97 | 1.00 |
| RUQ X LUQ | 1.01 | 0.99 | 1.03 |
| RLQ X RUQ X LUQ | 1.00 | 1.00 | 1.00 |
| LLQ X LUQ | 1.02 | 1.00 | 1.04 |
| RLQ X LLQ X LUQ | 1.00 | 1.00 | 1.00 |
| RUQ X LLQ X LUQ | 1.00 | 1.00 | 1.00 |
| RLQ X RUQ X LLQ | 1.00 | 1.00 | 1.00 |
| X LUQ | | | |

TABLE 3
device performance in derivation sample

| | Total | No imaging or | Patient required imaging |
|---|---|---|---|
| | N (column | admission | or admission |
| Device prediction | %) | N (row %) | N (row %) |
| Low | 2 (3%) | 2 (100%) | 0 (0%) |
| Medium | 21 (28%) | 8 (38%) | 13 (62%) |
| High | 53 (70%) | 0 (0%) | 53 (100%) |

P value for Fisher's exact test <0.001

In an initial feasibility study as described above, complete data was collected on 76 patients presenting to a large county hospital emergency department with chief complaint of abdominal-related complaints: pain, nausea, vomiting and/or diarrhea. Patients with abdominal pain who need unscheduled care may choose to use telehealth if it were possible to care for abdominal pain virtually. Currently, these patients must choose between an urgent care or an emergency department to get their care, so a sample of ED patients with abdominal pain may be a reasonable representation of patients that could benefit from virtual care alternatives for which embodiments of the disclosed palpation device may be employed. In this sample, patients were asked to use the device on themselves. Separately, a blinded physician would evaluate the patient as per the standard of care, including palpation of the abdomen. In this sample, the device was able to correctly categorize patients into low, indeterminate (medium), and high-risk groups for the physician ordering advanced imaging or hospital admission. 100% (53/53) of the patients that the device predicted as high risk required imaging and/or admission, which suggests the device is very effective at detecting high acuity pathologies. 13/21 patients (61.9%) in the indeterminate-risk group underwent imaging and/or admission. Indeterminate risk readings were, therefore, unable to predict the need for imaging/admission reliably. None of the patients (0/2) deemed as low risk required imaging and/or admission.

In some embodiments, the device is an aid in assessing the likelihood that a patient with abdominal pain being assessed virtually through telehealth requires in-person imaging or hospital admission. Thus, there may be two types of errors that occur with use of the device: falsely recommending an in-person visit when it is not needed (i.e., a false positive), or falsely recommending virtual care when an in-person visit is needed (i.e., a false negative).

If the device unnecessarily recommends an in-person visit, the patient will receive the current treatment standard and not be exposed to potential risk to health. The patient may experience some lost time and/or temporary anxiety until they are seen in-person by a healthcare provider. Upon in-person examination, a physician would palpate the patient's abdomen and if needed, order imaging or admit the patient. Thus, a false positive result would not present an increased risk to health over the current standard of care.

On the other hand, if the device indicates that a patient is at low or indeterminate risk when the patient has a surgical abdomen, there is the potential for a delay in treatment. This risk will be mitigated by the labeling of the device, which will indicate that the results from the device must be assessed together with a patient's clinical signs and symptoms and the device results should never be used to override clinical judgment if a patient's signs and symptoms indicate that a patient should be seen in-person for imaging or hospital admission. Further, patients are currently being assessed virtually for abdominal pain. This assessment relies entirely on qualitative self-reports from patients. In some embodiments, the proposed device can provide additional information beyond what virtual providers have today. Thus, the risk of a potential false negative is no greater than the potentially unreliable self-report from a patient that is provided today via telehealth. The risk of potential false positives and false negative results through use of embodiments of the present invention can be appropriately mitigated through additional bench, usability, and clinical studies.

From a technological perspective, the act of using embodiments of the disclosed the palpation device itself, does not have the potential to cause a risk to health. Just as if a physician were pushing on a patient's belly, the pressing will only occur up to the point of the patient experiencing pain provided the patient and/or user performs the palpations using the palpation device as instructed. Thus, from a technological perspective, the risks to health are low and can be addressed through appropriate verification and validation testing, including software, electrical safety, and electromagnetic compatibility testing.

Embodiments of the current invention disclose methods that enable a range of uses including a fully remote use case with a patient using the palpation device, and a remote care provider via telehealth. If there is no escalation in care needed (e.g., admission to the ER or advanced imaging), the patient care could also be fully self-service. Other care locations may also be contemplated in some embodiments discussed herein, including pharmacies, clinics, hospitals, or even schools, work sites, etc. Potential users could be students, customers or employees doing self-palpation, or palpation with a care provider such as a physician (MD), a registered nurse (RN), an nurse practitioner (NP), a pharmacy technician, or a school nurse, but are by no means limited by the examples above.

The value proposition to the patient of embodiments of the present disclosure include not having to make unnecessary trips to the emergency department (ED), and reduced cost through receiving the correct level of care.

The value proposition to insurance companies of embodiments of the present disclosure include faster and more accurate triage, avoiding imaging and unnecessary surgery costs, while improving patient outcomes, including reduced hospital and ED admissions, readmissions, and emergency surgeries.

The value proposition to employers of embodiments of the present disclosure could include reducing missed work days by employees, and reduced care costs (especially for self-insured employers).

Finally, the value proposition to physicians and care providers of embodiments of the present disclosure include an increase in workflow throughput, better care metrics and results, and reduced liability to patients.

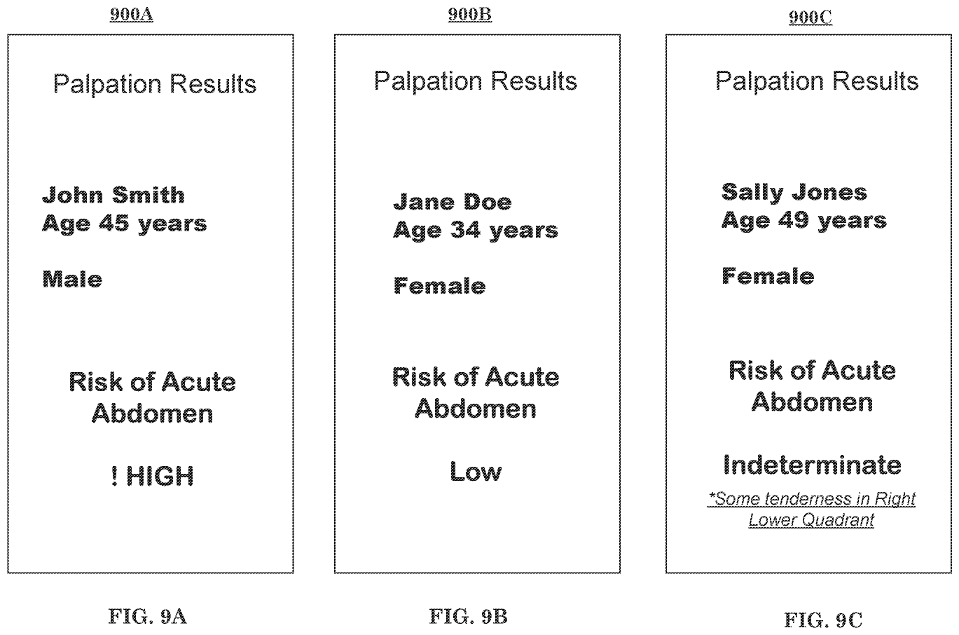

FIGS. 9A-9C illustrate exemplary wireframe diagrams 900A, 900B, and 900C respectively depicting screenshots showing an exemplary user interface for a clinician treating a patient using a medical palpation device for remote physical examination in accordance with an embodiment of the present disclosure. In screenshot diagram 900A, an exemplary screen of a smart phone or other mobile device of a clinician or physician is shown depicting the palpation results for a 45 year old male patient, John Smith, who has just completed the self-palpation process. The software analytics process has been completed and a risk of acute abdomen is given as “HIGH” meaning that clinician advises the patient to go to the ED and/or orders advanced imaging tests.

In screenshot diagram 900B, an exemplary screen of a smart phone or other mobile device of a clinician or physician is shown depicting the palpation results for a 34 year old female patient, Jane Doe, who has just completed the self-palpation process. The software analytics process has been completed and a risk of acute abdomen is given as “LOW” meaning that depending on the patient's history, the clinician does not need to advise the patient to go to the ED and/or order further imaging tests. In screenshot diagram 900C, an exemplary screen of a smart phone or other mobile device of a clinician or physician is shown depicting the palpation results for a 49 year old female patient, Sally Jones, who has just completed the self-palpation process. The software analytics process has been completed and a risk of acute abdomen is given as “Indeterminate”, and a further note was included that there was some tenderness in the Right Lower Quadrant of the abdomen. At this point the clinician may review the patient's history more closely or conduct further examination via telehealth, and then depending if further concerns are raised, the clinician may advise the patient to go to the ED and/or order further imaging tests.

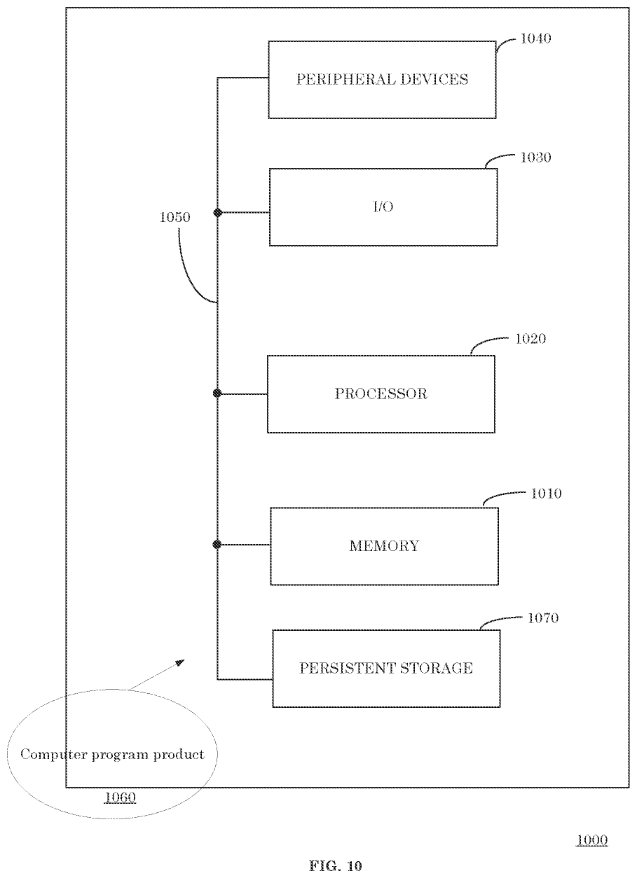

FIG. 10 shows an example of a computer system 1000, one or more of which may be used to implement one or more of the apparatuses, systems, and methods illustrated herein, such as devices 100-600 and method 800 discussed herein, and user interfaces 700A-700F and 900A-900C. Computer system 1000 executes instruction code contained in a computer program product 1060. Computer program product 1060 comprises executable code in an electronically readable medium that may instruct one or more computers such as computer system 1000 to perform processing that accomplishes the exemplary method steps performed by the embodiments referenced herein.

The electronically readable medium may be any non-transitory medium that stores information electronically and may be accessed locally or remotely, for example via a network connection. In alternative embodiments, the medium may be transitory. The medium may include a plurality of geographically dispersed media each configured to store different parts of the executable code at different locations and/or at different times. The executable instruction code in an electronically readable medium directs the illustrated computer system 1000 to carry out various exemplary tasks described herein. The executable code for directing the carrying out of tasks described herein would be typically realized in software. However, it will be appreciated by those skilled in the art, that computers or other electronic devices might utilize code realized in hardware to perform many or all the identified tasks without departing from the present invention. Those skilled in the art will understand that many variations on executable code may be found that implement exemplary methods within the spirit and the scope of the present invention.

The code or a copy of the code contained in computer program product 1060 may reside in one or more storage persistent media (not separately shown) communicatively coupled to system 1000 for loading and storage in persistent storage device 1070 and/or memory 1010 for execution by processor 1020. Computer system 1000 also includes I/O subsystem 1030 and peripheral devices 1040. I/O subsystem 1030, peripheral devices 1040, processor 1020, memory 1010, and persistent storage device 1070 are coupled via bus 1050. Like persistent storage device 1070 and any other persistent storage that might contain computer program product 1060, memory 1010 is a non-transitory media (even if implemented as a typical volatile computer memory device). Moreover, those skilled in the art will appreciate that in addition to storing computer program product 1060 for carrying out processing described herein, memory 1010 and/or persistent storage device 1070 may be configured to store the various data elements referenced and illustrated herein.

Those skilled in the art will appreciate computer system 1000 illustrates just one example of a system in which a computer program product in accordance with an embodiment of the present invention may be implemented. To cite but one example of an alternative embodiment, execution of instructions contained in a computer program product in accordance with an embodiment of the present invention may be distributed over multiple computers, such as, for example, over the computers of a distributed computing network.

Instructions for implementing a machine learning-based propensity score model, a logistic regression model, a probit model, and/or an artificial neural network implementing any of the above in accordance with disclosed embodiments may reside in computer program product 1060. When processor 1020 is executing the instructions of computer program product 1060, the instructions, or a portion thereof, are typically loaded into working memory 1010 from which the instructions are readily accessed by processor 1020.

In one embodiment, processor 1020 in fact comprises multiple processors which may comprise additional working memories (additional processors and memories not individually illustrated) including one or more graphics processing units (GPUs) comprising at least thousands of arithmetic logic units supporting parallel computations on a large scale. GPUs are often utilized in deep learning applications because they can perform the relevant processing tasks more efficiently than can typical general-purpose processors (CPUs). Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 1020 may be implemented as one or more general purpose processors (preferably having multiple cores) without necessarily departing from the spirit and scope of the present invention.

### Exemplary Palpation Device Embodiments

1. A handheld palpation device for medical examination, comprising:
   a. a handle having a rounded shape configured for a hand of a user to hold while using the handheld palpation device to apply pressure to the user's body, wherein the handle includes an interior space and an opening to the interior space;
   b. a palpation head having a first end and a second end, wherein the first end is configured to fit inside the opening and the second end comprises a round pliable head configured to contact the body of the user when pressure is applied by the user;
   c. a pressure pad positioned in the interior space of the handle having a first surface configured to contact the first end of the palpation head wherein the pressure pad captures electrical pressure data when pressure is applied by the user;
   d. a sensor chip positioned in the interior space of the handle and connected to the pressure pad, wherein the sensor chip is configured to receive the electrical pressure data from the pressure pad and calculate one or more quantitative pressure measurements;
   e. a communications chip positioned in the interior space of the handle and connected to the sensor chip, wherein the communications chip is configured to receive the quantitative pressure measurements from the sensor chip and communicate the quantitative pressure measurements to an external device;
   f. a power source connected to the sensor chip and the communications chip; and
   g. a lock system configured to hold the first end of the palpation head in the interior space of the handle.
2. The palpation device of embodiment 1, wherein the communications chip comprises a Bluetooth connection.
3. The palpation device of embodiment 1, wherein the sensor chip comprises a voltage divider circuit.
4. The palpation device of embodiment 1, wherein the pressure pad comprises a force-sensitive resistor.
5. The palpation device of embodiment 1, wherein the lock system comprises one or more pins on a surface of the palpation head between the first and second ends, wherein the one or more pins are configured to reside inside the interior surface of the handle;
6. The palpation device of embodiment 1, wherein the first end of the palpation head comprises a compression member that supports pressure applied by the user holding the palpation device.
7. The palpation device of embodiment 6, wherein the compression member comprises a rigid three-dimensional object.
8. The palpation device of embodiment 6, wherein the compression member comprises a spring.
9. The palpation device of embodiment 1, wherein the power source comprises a battery located inside the handle.
10. The palpation device of embodiment 1, further comprising a cord connection connected to the power source.
11. The palpation device of embodiment 1, further comprising a power cord having a first end connected to the cord connection.
12. The palpation device of embodiment 11, wherein the power cord comprises a second end having one or more adapters configured for connection to a user device.
13. The palpation device of embodiment 12, wherein the one or more adapters comprises a USB-C connector.
14. The palpation device of embodiment 1, wherein the palpation head comprises a massage head.
15. The palpation device of embodiment 1, wherein the handle comprises rigid plastic.

### Exemplary Palpation Device Software Embodiments

16. A method of conducting a remote medical examination of a patient for abdominal pain, the method comprising:
   a. displaying a set of palpation instructions to a patient on a user device, wherein the set of palpation instructions guide the patient to palpate a plurality of locations on the patient's body using a handheld palpation device wherein the plurality of locations includes a baseline location;
   b. receiving a set of pressure readings from the handheld palpation device for each of the plurality of locations of the patient's body;
   c. calculating an average pressure reading for the set of pressure readings for each of the plurality of locations of the patient's body, including the baseline location;
   d. calculating an maximum reliable pressure reading for at least a subset of the plurality of locations of the patient's body, wherein the maximum reliable pressure reading is a difference between the average pressure reading for the location and the average pressure reading for the baseline location; and
   e. providing a recommendation to the patient or a health care provider of the patient for further medical treatment, wherein the recommendation is derived based on the calculated maximum reliable pressure readings, and the recommendation comprises one or more of imaging, admission to an emergency department, or virtual medical treatment.
17. The method of embodiment 16, wherein the plurality of locations comprises a right upper quadrant of an abdomen, a right lower quadrant of the abdomen, a left upper quadrant of the abdomen, a left lower quadrant of the abdomen, and a sternum.
18. The method of embodiment 16, wherein the baseline location comprises the sternum.
19. The method of embodiment 16, wherein the recommendation is derived using multiple logistic regression, wherein an outcome variable comprises whether the patient requires imaging or admission to the emergency department.
20. The method of embodiment 19, wherein a plurality of predictor variables comprises the maximum reliable pressures for the right upper quadrant, the right lower quadrant, the left upper quadrant, and the right lower quadrant.
21. The method of embodiment 19, wherein the recommendation further comprises assigning a patient requiring imaging or admission to the emergency department to a low, medium, or high risk category.
22. The method of embodiment 16, wherein the handheld palpation device comprises any one of embodiments 1-15.
23. The method of embodiment 16, wherein the user device comprises a mobile phone, a tablet, a laptop computer, or a desktop computer.
24. A non-transitory computer-readable medium storing one or more software instructions, which upon execution by a processor, perform the following operations:
   a. displaying a set of palpation instructions to a patient on an external device, wherein the set of palpation instructions guide the patient to palpate a plurality of locations on the patient's body using a handheld palpation device wherein the plurality of locations includes a baseline location;
   b. receiving a set of pressure readings from the handheld palpation device for each of the plurality of locations of the patient's body;
   c. calculating an average pressure reading for the set of pressure readings for each of the plurality of locations of the patient's body, including the baseline location;
   d. calculating a maximum reliable pressure reading for at least a subset of the plurality of locations of the patient's body, wherein the maximum reliable pressure reading is a difference between the average pressure reading for the location and the average pressure reading for the baseline location; and
   e. providing a recommendation to the patient or a health care provider of the patient for further medical treatment, wherein the recommendation is derived based on the calculated maximum reliable pressure readings, and the recommendation comprises one or more of imaging, admission to an emergency department, or virtual medical treatment.
25. The palpation device of embodiment 1, wherein the external device comprises a mobile phone, a tablet, a laptop computer, or a desktop computer.

While the present invention has been particularly described with respect to the illustrated embodiments, it will be appreciated that various alterations, modifications, and adaptations may be made based on the present disclosure and are intended to be within the scope of the present invention. While the invention has been described in connection with what are presently considered to be the most practical and preferred embodiments, it is to be understood that the present invention is not limited to the disclosed embodiments but, on the contrary, is intended to cover various modifications and equivalent arrangements included within the scope of the underlying principles of the invention as described by the various embodiments referenced above and below.

1. A handheld palpation device for medical examination, comprising:
a handle comprising a rounded shape to hold while using the handheld palpation device to apply pressure to a user's body, wherein the handle includes an interior space and an opening to the interior space;
a palpation head having a first end and a second end, wherein the first end is configured to fit inside the opening and the second end comprises a round pliable head configured to contact the body of the user when pressure is applied;
a pressure pad positioned in the interior space of the handle having a first surface configured to contact the first end of the palpation head wherein the pressure pad captures electrical pressure data when pressure is applied;
a sensor chip positioned in the interior space of the handle and connected to the pressure pad, wherein the sensor chip is configured to receive the electrical pressure data from the pressure pad and calculate one or more quantitative pressure measurements;
a communications chip positioned in the interior space of the handle and connected to the sensor chip, wherein the communications chip is configured to receive the quantitative pressure measurements from the sensor chip and communicate the quantitative pressure measurements to an external device;
a power source connected to the sensor chip and the communications chip; and
a lock system configured to hold the first end of the palpation head in the interior space of the handle.

2. The palpation device of claim 1, wherein the communications chip comprises a Bluetooth connection.

3. The palpation device of claim 1, wherein the sensor chip comprises a voltage divider circuit.

4. The palpation device of claim 1, wherein the pressure pad comprises a force-sensitive resistor.

5. The palpation device of claim 1, wherein the lock system comprises one or more pins on a surface of the palpation head between the first and second ends, wherein the one or more pins are configured to reside inside the interior surface of the handle.

6. The palpation device of claim 1, wherein the first end of the palpation head comprises a compression member that supports pressure applied by the user holding the palpation device.

7. The palpation device of claim 6, wherein the compression member comprises a rigid three-dimensional object.

8. The palpation device of claim 6, wherein the compression member comprises a spring.

9. The palpation device of claim 1, wherein the power source comprises a battery located inside the handle.

10. The palpation device of claim 1, further comprising a cord connection connected to the power source.

11. The palpation device of claim 1, further comprising a power cord having a first end connected to the cord connection.

12. The palpation device of claim 11, wherein the power cord comprises a second end having one or more adapters configured for connection to a user device.

13. The palpation device of claim 12, wherein the one or more adapters comprises a USB-C connector.

14. The palpation device of claim 1, wherein the palpation head comprises a massage head.

15. The palpation device of claim 1, wherein the handle comprises rigid plastic.

16. A method of conducting a remote medical examination of a patient for abdominal pain, the method comprising:
displaying a set of palpation instructions to a patient on a user device, wherein the set of palpation instructions guide the patient to palpate a plurality of locations on the patient's body using a handheld palpation device wherein the plurality of locations includes a baseline location;
receiving a set of pressure readings from the handheld palpation device for each of the plurality of locations of the patient's body;
calculating an average pressure reading for the set of pressure readings for each of the plurality of locations of the patient's body, including the baseline location;
calculating a maximum reliable pressure reading for at least a subset of the plurality of locations of the patient's body, wherein the maximum reliable pressure reading is a difference between the average pressure reading for the location and the average pressure reading for the baseline location; and
providing a recommendation to the patient or a health care provider of the patient for further medical treatment, wherein the recommendation is derived based on the calculated maximum reliable pressure readings, and the recommendation comprises one or more of imaging, admission to an emergency department, or virtual medical treatment.

17. The method of claim 16, wherein the plurality of locations comprises a right upper quadrant of an abdomen, a right lower quadrant of the abdomen, a left upper quadrant of the abdomen, a left lower quadrant of the abdomen, and a sternum.

18. The method of claim 16, wherein the baseline location comprises the sternum.

19. The method of claim 16, wherein the recommendation is derived using multiple logistic regression, wherein an outcome variable comprises whether the patient requires imaging or admission to the emergency department.

20. The method of claim 19, wherein a plurality of predictor variables comprises the maximum reliable pressures for the right upper quadrant, the right lower quadrant, the left upper quadrant, and the right lower quadrant.

21. The method of claim 19, wherein the recommendation further comprises assigning the patient to a low, indeterminate, or high risk category.

22. The method of claim 16, wherein the handheld palpation device comprises:
a handle comprising a rounded shape to hold while using the handheld palpation device to apply pressure to the patient's body, wherein the handle includes an interior space and an opening to the interior space;
a palpation head having a first end and a second end, wherein the first end is configured to fit inside the opening and the second end comprises a round pliable head configured to contact the body of the patient when pressure is applied;
a pressure pad positioned in the interior space of the handle having a first surface configured to contact the first end of the palpation head wherein the pressure pad captures electrical pressure data when pressure is applied;
a sensor chip positioned in the interior space of the handle and connected to the pressure pad, wherein the sensor chip is configured to receive the electrical pressure data from the pressure pad and calculate one or more quantitative pressure measurements;
a communications chip positioned in the interior space of the handle and connected to the sensor chip, wherein the communications chip is configured to receive the quantitative pressure measurements from the sensor chip and communicate the quantitative pressure measurements to an external device;
a power source connected to the sensor chip and the communications chip; and
a lock system configured to hold the first end of the palpation head in the interior space of the handle.

23. The method of claim 16, wherein the user device comprises a mobile phone, a tablet, a laptop computer, or a desktop computer.

24. A non-transitory computer-readable medium storing one or more software instructions, which upon execution by a processor, perform the following operations:
displaying a set of palpation instructions to a patient on an external device, wherein the set of palpation instructions guide the patient to palpate a plurality of locations on the patient's body using a handheld palpation device wherein the plurality of locations includes a baseline location;
receiving a set of pressure readings from the handheld palpation device for each of the plurality of locations of the patient's body;
calculating an average pressure reading for the set of pressure readings for each of the plurality of locations of the patient's body, including the baseline location;
calculating an maximum reliable pressure reading for at least a subset of the plurality of locations of the patient's body, wherein the maximum reliable pressure reading is a difference between the average pressure reading for the location and the average pressure reading for the baseline location; and
providing a recommendation to the patient or a health care provider of the patient for further medical treatment, wherein the recommendation is derived based on the calculated maximum reliable pressure readings, and the recommendation comprises one or more of imaging, admission to an emergency department, or virtual medical treatment.

25. The palpation device of claim 1, wherein the external device comprises a mobile phone, a tablet, a laptop computer, or a desktop computer.
